SEQUENCE LISTING (4 sequences)

SEQ ID NO: 1 - RNA, 21 nt, Artificial Sequence
  Sense strand of siRNA duplex against HGK (ZCI) ORF
aagaagaggaggaagugccug

SEQ ID NO: 2 - RNA, 21 nt, Artificial Sequence
  Sense strand of siRNA duplex against HGK (ZCI) ORF
aacacauauggaaggaucacc

SEQ ID NO: 3 - DNA, 19 bp, Artificial Sequence
  Scrambled HGK siRNA sequence
gcgcgctttgtaggattcg

SEQ ID NO: 4 - DNA, 19 bp, Artificial Sequence
  LaminAC siRNA sequence
ctggacttccagaagaaca

(19) United States
(12) Patent Application Publication
(10) Pub. No.: US 2005/0276757 A1
(43) Pub. Date: Dec. 15, 2005

(54) Methods of using ZC1 and ZC3 kinase substrate phosphorylation as biomarkers

(75) Inventors: Jocelyn H. Wright, Pacifica, CA (US); Victoria Sung, San Francisco, CA (US); Michel Paul Faure, Oakland, CA (US); Elizabeth Marie Blackwood, San Carlos, CA (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 11/128,106

(22) Filed: May 11, 2005

Related U.S. Application Data
(60) Provisional application No. 60/570,083, filed on May 12, 2004.

(51) Int. Cl. A61K 49/00; C12Q 1/48

(52) U.S. Cl. 424/9.2; 435/15

(57) ABSTRACT

The present invention provides novel biomarkers for cancer: the phosphorylation of ZC1 and/or ZC3 kinase substrates, particularly p120 catenin and Abi-1. Accordingly, the invention provides methods of determining whether a compound modulates ZC1 or ZC3 kinase activity, methods of determining whether a mammal is responding to a compound that modulates ZC1 or ZC3 kinase activity, and methods of predicting whether a mammal will respond therapeutically to a method of treating cancer by administering a modulator of ZC1 or ZC3 kinase.

### BACKGROUND OF THE INVENTION

Biomarkers are molecular indicators of biological events, or phenomena, in organisms. Changes in the level of a biomarker can evidence an organism's biological response to a stimulus, such as a chemical compound. The biological responses may include events at a molecular, cellular or whole organism level.

Measured changes in biomarker levels are useful to gauge whether or not a particular effect has been achieved, or is likely to be achieved, in an organism. Thus, changes in biomarker levels can indicate whether or not a compound induces a particular biological response in an organism. Similarly, changes in biomarker levels can indicate that an organism has been exposed to a particular compound. In a clinical context, changes in biomarker levels can indicate whether an organism is experiencing, or will experience, a therapeutic effect or toxic event in response to a compound.

In the study and treatment of cancer, a continuing need exists for identification of new biomarkers. Cancer-related biomarkers can be employed to identify new effective cancer treatments, to monitor patient tolerance and/or therapeutic response to a cancer treatment, and to predict effective cancer treatments for individual patients.

### SUMMARY OF THE INVENTION

The present inventors have discovered endogenous substrates for ZC1 and ZC3 kinases, both of which promote cell transformation and tumor growth. According to the invention, the preferred substrate of ZC1 is p120 catenin and the preferred substrate of ZC3 is Abi-1. Phosphorylation of the ZC1 and ZC3 substrates constitutes a new biomarker for cancer.

Thus, the present invention provides methods of determining whether a test compound modulates ZC1 kinase activity in a mammal. In one embodiment, the determination of whether a compound modulates ZC1 kinase activity in a mammal involves the following steps:
(a) measuring a base level of phosphorylation of a ZC1 kinase substrate in the mammal,
(b) exposing the mammal to a compound, then
(c) measuring a post-exposure level of phosphorylation of the ZC1 kinase substrate in the mammal, and
(d) comparing the base level with the post-exposure level.
   A difference in the base level and post-exposure level indicates that the compound modulates ZC1 kinase activity.

In another embodiment, the determination of whether a compound modulates ZC1 kinase activity in a mammal involves the following steps:
(a) exposing a mammal to a compound, then
(b) measuring a post-exposure level of phosphorylation of a ZC1 kinase substrate in the mammal, and
(c) comparing the post-exposure level of phosphorylation with a standard level of phosphorylation for the mammal.
   A difference between the post-exposure level and the standard level indicates that the compound modulates ZC1 kinase activity.

In another aspect, the invention provides a method of determining whether a mammal is responding to a compound that modulates ZC1 kinase activity. This involves comparing a base level of ZC1 substrate phosphorylation in a mammal against a level of ZC1 substrate phosphorylation after the mammal is exposed to a compound that modulates ZC1 kinase activity. A difference in the base and post-exposure levels indicates that the mammal is responding to the compound. Preferably, phosphorylation levels are derived from tumor samples in the mammal, and therefore can indicate whether therapy with a ZC1 kinase modulator is operating effectively.

In yet another aspect, the invention provides methods of predicting whether a mammal with cancer will respond therapeutically to administration of a modulator of ZC1 kinase. The method of predicting involves (a) measuring the level of ZC1 substrate phosphorylation in a mammal with cancer, and (b) comparing the level of ZC1 substrate phosphorylation in the mammal against a standard level of ZC1 substrate phosphorylation for the mammal. A difference in the phosphorylation levels indicates that the mammal will respond therapeutically to a modulator of ZC1 kinase. Preferably, the phosphorylation level is measured in a tumor sample.

The present invention also provides methods for determining whether a compound modulates ZC3 kinase activity in a mammal. In one embodiment, this determination can be made by performing the following steps:
(a) measuring a base level of phosphorylation of a ZC3 kinase substrate in the mammal,
(b) exposing the mammal to a compound, then
(c) measuring a post-exposure level of Abi-1 phosphorylation in the mammal, and
(d) comparing the base level with the post-exposure level.
   A difference in the base level and post-exposure level indicates that the compound modulates ZC3 kinase activity.

In another embodiment, the determination of whether a compound modulates ZC3 kinase activity in a mammal involves the following steps:
(a) exposing a mammal to a compound, then
(b) measuring a post-exposure level of phosphorylation of a ZC3 kinase substrate in the mammal, and
(c) comparing the post-exposure level of phosphorylation with a standard level of phosphorylation for the mammal.
   A difference between the post-exposure level and the standard level indicates that the compound modulates ZC3 kinase activity.

In another aspect, the invention provides a method of determining whether a mammal is responding to a compound that modulates ZC3 kinase activity. This involves comparing a base level of ZC3 substrate phosphorylation in a mammal against a level of ZC3 substrate phosphorylation after the mammal is exposed to a compound that modulates ZC3 kinase activity. A difference in the base and post-exposure levels indicates that the mammal is responding to the compound. Preferably, phosphorylation levels are derived from tumor samples in the mammal, and therefore can indicate whether therapy with a ZC3 kinase modulator is operating effectively.

In yet another aspect, the invention includes methods of predicting whether a mammal with cancer will respond therapeutically to administration of a modulator of ZC3 kinase. The method of predicting involves (a) measuring the level of ZC3 substrate phosphorylation in a mammal with cancer, and (b) comparing the level of ZC3 substrate phosphorylation in the mammal against a standard level of ZC3 substrate phosphorylation for the mammal. A difference in the phosphorylation levels indicates that the mammal will respond therapeutically to a modulator of ZC3 kinase. Preferably, the phosphorylation level is measured in a tumor sample.

### BRIEF DESCRIPTION OF THE DRAWINGS

FIG. 1 shows immunofluorescence results demonstrating that ZC1 and p120 catenin co-localize to adherins (cell-cell) junctions in two cell lines: H1299 non-small cell lung cancer cells and RIE rat intestinal epithelial cells. The top panels show staining of ZC1, the middle panels show staining of p120 catenin, and the bottom panels show co-staining of ZC1 and p120 catenin.

FIG. 2 shows the results of immunoprecipitation reactions demonstrating that endogenous ZC1 and p120 catenin co-associate in cell extracts of H1299 and RIE cells, indicating a shared function in the cells.

FIG. 3 shows the results of experiments demonstrating that ZC1 phosphorylates p120 catenin in vitro. p120 catenin was isolated from cells via immunoprecipitation, using anti-p120 antibody. Purified recombinant ZC1 enzyme phosphorylated the isolated p120 catenin, as shown in the autoradiograms of the lower 2 panels. Additionally, p120 catenin immunoprecipitated from cells co-expressing ZC1 exhibited in vitro phosphorylation by associated ZC1, which co-immunoprecipitated out of the cell extract. A gel mobility shift in the stained p120 catenin protein band (top panel) indicates that p120 catenin was quantitatively phosphorylated by ZC1 in vitro, meaning that p120 catenin is a very efficient substrate for ZC1 kinase.

FIG. 4 shows the results of experiments demonstrating that ZC1 phosphorylates p120 catenin in vivo. The phosphorylation state of p120 catenin in cells was examined using a phospho-threonine (pThr) antibody. A plasmid encoding p120 protein was co-transfected into 293T cells alone and in combination with 8 different forms of ZC1, including active and inactive forms of the enzyme. When immunoprecipitated from cell extracts, p120 catenin reactivity with the pThr antibody increased in those extracts prepared from cells also transfected with active forms of ZC1 (top panel, lanes 2, 5, 6 and 8 from the left) as compared to cells transfected with p120 catenin alone (first lane on the left), or cells co-expressing inactive forms of ZC1 (lanes 4 and 7). Cells expressing truncated forms of ZC1 were less effective at increasing p120 reactivity with pThr antibody than full length forms (lanes 9 and 10 from the left), but still significantly higher than inactive forms of the enzyme. Additionally, co-transfected ZC1 protein associated with the isolated p120 catenin, corroborating the data in FIG. 2 (anti-ZC1 blot shown in middle panel). Total levels of p120 catenin protein in the immune complex are shown by anti-p120 western blot (bottom panel).

FIG. 5 shows the results of a phage display screen demonstrating that the SH3 domains of Abi-1 and Abi-2 both interact with the linker region of ZC3 (aa 672-1032). This region of ZC3 contains several PXXP motifs that likely account for the binding.

FIG. 6 shows the results of experiments demonstrating that ZC3 interacts with Abi-1 and Abi-2B in cells. When ZC3 or ZC3B, a splice variant of ZC3, was co-transfected with Abi-1 or Abi-2B into COS-7 cells, ZC3 co-immunoprecipitated with the Abi proteins, and vice versa. Panel A shows that Abi-2B co-immunoprecipitates with ZC3 and ZC3B when they are co-expressed in cells. Abi-1 is not visible because it runs too close to the IgG heavy chain band (thick band across the blot). Panel B shows that both ZC3 and ZC3B co-immunoprecipitate with Abi-1 (top blot) and that ZC3 co-immunoprecipitates with Abi-2B (second blot from the top).

FIG. 7 shows the results of experiments demonstrating that Abi-1 immunoprecipitated from 293T cells is phosphorylated in vitro by the kinase domain of ZC3 fused to GST. A strongly phosphorylated band corresponding to Abi-1 appears in the sample containing immunoprecipitated Abi-1 and GST-ZC3KD. The weaker band in the other lanes corresponds to GST-ZC3KD itself; its expected size is 63 KD. MBP is known to be a good in vitro substrate for ZC3 and is indeed strongly phosphorylated.

FIG. 8 shows the results of experiments demonstrating that over-expression of wild-type HGK (ZC1) potentiates growth of H1299 lung carcinoma cells in soft agar. (A) An anti-myc tag western blot shows relative HGK protein levels in isolated clones; (B) Photomicrographs of clones grown in soft agar in 10% serum are shown; (C) Relative number of colonies per plate for each clone (average of 3 plates graphed, with error bars indicating the standard deviation) is shown.

FIG. 9 shows the results of experiments demonstrating that HGK (ZC1) kinase activity is required for potentiation of anchorage-independent growth of H1299 lung carcinoma cells. (A) An anti-HGK western blot shows relative expression of HGK in vector, wild type, and kinase-inactive (KR) in isolate clones; (B) Photographs show representative plates containing soft agar colonies form the different clones; (C) Relative number of colonies per plate for each clone (average of 2 plates graphed, with error bars indicating the range); (D) Relative growth of clones grown in monolayer in 10% and 0.5% serum is shown.

FIG. 10 shows the results of experiments demonstrating a reduction of endogenous HGK (ZC1) kinase via RNAi reduced anchorage-independent growth in (A) Hela and (B) A549 tumor cell lines. The top panels in each of A and B show anti-HGK western blots after transfection with HGK oligos vs. a scrambled version of the oligos and oligofectamine alone. The bottom left panels show photomicrographs of colonies grown in soft agar after transfection. The lower right panels show graphs of the relative number of colonies per plate. Quantification from 2 plates per transfection are shown.

FIG. 11 shows the results of experiments demonstrating that HGK (ZC1) kinase activity potentates growth of H1299 cells in mouse xenografts. (A) Results of anti-myc IP kinase assays performed on lysates from the same clones analyzed in vivo, with myelin basic protein (MBP) as a substrate are shown; (B) A graph shows the average tumor volume (8-10 animal averaged for each clone) as a function of time; (C) Anti-myc tag western blots of anti-myc tag IP from tumor lysates show that HGKmyc expression is retained in excised tumor at the end of the experiment (data for Vector-1 and Wild type-8 tumor are shown).

FIG. 12 is a table showing a summary of xenograft tumor data for clones.

FIG. 13 shows the results of experiments demonstrating that over-expression of active HGK (ZC1) induced cell rounding and a loss of adherins junctions in stable H1299 clones. (A) An anti-myc western blot shows relative HGK protein expression in clones; (B) Fluorescent light micrographs show vector and wild type expressing clones stained with FITC-conjugated anti-HGK antibody.

FIG. 14 shows the results of experiments demonstrating that infection of 3 tumor cell lines with HGK WT and KR adenovirus affects morphology and adhesion. (A) An anti-myc tag western blot shows the over-expressed HGK proteins, (B) Phase-contrast photomicrographs show cell morphology of infected cells 48 hours post-infection.

### DETAILED DESCRIPTION OF THE INVENTION

The present inventors have discovered endogenous substrates for ZC1 and ZC3 kinases, both of which promote cell transformation and tumor growth. According to the invention, the preferred substrate of ZC1 is p120 catenin and the preferred substrate of ZC3 is Abi-1. Phosphorylation of the ZC1 and ZC3 substrates constitutes a new biomarker for cancer.

In accord with the inventors' discovery, the present invention includes methods for determining whether a test compound modulates ZC1 or ZC3 kinase activity in a mammal. It also includes methods of determining whether a mammal is biologically responding to a compound that modulates ZC1 or ZC3 kinase activity. Further, the invention includes methods of accurately predicting whether a mammal with cancer will respond therapeutically to administration of a modulator of either ZC1 or ZC3 kinase.

In one embodiment of the present invention, the determination of whether a compound modulates ZC1 kinase activity in a mammal involves the following steps:
(a) measuring a base level of phosphorylation of a ZC1 kinase substrate in the mammal,
(b) exposing the mammal to a compound, then
(c) measuring a post-exposure level of phosphorylation of the ZC1 kinase substrate in the mammal, and
(d) comparing the base level with the post-exposure level.
   A difference in the base level and post-exposure level indicates that the compound modulates ZC1 kinase activity.

In another embodiment, the determination of whether a compound modulates ZC1 kinase activity in a mammal involves the following steps:
(a) exposing a mammal to a compound, then
(b) measuring a post-exposure level of phosphorylation of a ZC1 kinase substrate in the mammal, and
(c) comparing the post-exposure level of phosphorylation with a standard level of phosphorylation for the mammal.
   A difference between the post-exposure level and the standard level indicates that the compound modulates ZC1 kinase activity.

In another aspect, the invention also provides a method of determining whether a mammal is responding to a compound that modulates ZC1 kinase activity. This involves comparing a base level of ZC1 substrate phosphorylation in a mammal against a level of ZC1 substrate phosphorylation after the mammal is exposed to a compound that modulates ZC1 kinase activity. A difference in the base and post-exposure levels indicates that the mammal is responding to the compound. Preferably, phosphorylation levels are derived from tumor samples in the mammal, and therefore can indicate whether therapy with a ZC1 kinase modulator is operating effectively.

In yet another aspect, the invention includes methods of predicting whether a mammal with cancer will respond therapeutically to administration of a modulator of ZC1 kinase. The method of predicting involves (a) measuring the level of ZC1 substrate phosphorylation in a mammal with cancer, and (b) comparing the level of ZC1 substrate phosphorylation in the mammal against a standard level of ZC1 substrate phosphorylation for the mammal. A difference in the phosphorylation levels indicates that the mammal will respond therapeutically to a modulator of ZC1 kinase. Preferably, the phosphorylation level is measured in a tumor sample.

The present invention also provides methods for determining whether a compound modulates ZC3 kinase activity in a mammal. In one embodiment, this determination can be made by performing the following steps:
(a) measuring a base level of phosphorylation of a ZC3 kinase substrate in the mammal,
(b) exposing the mammal to a compound, then
(c) measuring a post-exposure level of Abi-1 phosphorylation in the mammal, and
(d) comparing the base level with the post-exposure level.
   A difference in the base level and post-exposure level indicates that the compound modulates ZC3 kinase activity.

In another embodiment, the determination of whether a compound modulates ZC3 kinase activity in a mammal involves the following steps:
(a) exposing a mammal to a compound, then
(b) measuring a post-exposure level of phosphorylation of a ZC3 kinase substrate in the mammal, and
(c) comparing the post-exposure level of phosphorylation with a standard level of phosphorylation for the mammal.
   A difference between the post-exposure level and the standard level indicates that the compound modulates ZC3 kinase activity.

In another aspect, the invention provides a method of determining whether a mammal is responding to a compound that modulates ZC3 kinase activity. This involves comparing a base level of ZC3 substrate phosphorylation in a mammal against a level of ZC3 substrate phosphorylation after the mammal is exposed to a compound that modulates ZC3 kinase activity. A difference in the base and post-exposure levels indicates that the mammal is responding to the compound. Preferably, phosphorylation levels are derived from tumor samples in the mammal, and therefore can indicate whether therapy with a ZC3 kinase modulator is operating effectively.

In yet another aspect, the invention includes methods of predicting whether a mammal with cancer will respond therapeutically to administration of a modulator of ZC3 kinase. The method of predicting involves (a) measuring the level of ZC3 substrate phosphorylation in a mammal with cancer, and (b) comparing the level of ZC3 substrate phosphorylation in the mammal against a standard level of ZC3 substrate phosphorylation for the mammal. A difference in the phosphorylation levels indicates that the mammal will respond therapeutically to a modulator of ZC3 kinase. Preferably, the phosphorylation level is measured in a tumor sample.

ZC1 kinase, or HGK (hematopoietic progenitor kinase-like/germinal center kinase-like kinase), is a member of the human STE20/mitogen-activated protein kinase family of serine/threonine kinases. It is the ortholog of mouse NIK (Nck-interacting kinase). The structure of ZC1 and its relationship to other kinases are thoroughly described in U.S. patent application Ser. No. 09/291,417 (published Mar. 13, 2003) and Wright, J. H. et al., Mol. Cell. Biol., 23(6): 2068-82 (2003), both of which are incorporated herein by reference. Several splice variants of ZC1 exist.

ZC1 is understood to play a role in human cancers. More particularly, ZC1 functions in cellular adhesion, invasion and transformation. It is highly expressed in most tumor cell lines relative to normal tissue. Moreover, ZC1 plays an active role in transformation. This has been demonstrated in experiments showing that expression of inactive, dominant-negative mutants of ZC1 in both fibroblast and epithelial cell lines inhibits H-Ras-V12-induced focus formation. Additionally, expression of inactive mutant ZC1 inhibits anchorage-independent growth of cells, modulates integrin receptor expression and inhibits hepatocyte growth factor-stimulated epithelial cell invasion.

ZC3 also is a member of the human STE20/mitogen-activated protein kinase family of serine/threonine kinases. Its structure and relationship to other kinases are thoroughly described in U.S. patent application Ser. No. 09/291,417 (published Mar. 13, 2003), which is incorporated herein by reference. As with ZC1, several splice variants of ZC3 exist. ZC3 shares a high degree of homology with ZC1, and also is understood to play a role in human cancers. It is highly expressed in most breast cancers and leukemias, relative to normal tissues.

The present inventors have discovered that ZC1 kinase localizes to cell-cell junctions, where it associates with p120 catenin. Moreover, they have discovered that ZC1 kinase phosphorylates p120 catenin, both in vivo and in vitro. Indeed, p120 catenin constitutes a very efficient substrate for ZC1 kinase.

Thus, according to the present invention, p120 catenin constitutes a useful and preferred ZC1 kinase substrate. p120 catenin belongs to the Armadillo/β-catenin gene superfamily, originally described as a substrate for src and other receptor tyrosine kinases, and its expression is often altered or lost in tumors of the breast, prostate, colon, stomach, pancreas and bladder. Loss and change in localization of p120 is also associated with poor prognosis and advanced disease, suggesting a role in cancer progression.

In vivo, p120 catenin it is often localized to cellular adherins junctions, but also has been found in the cell nucleus, particularly in metastatic tumor cells that have lost cadherin expression. Tyrosine and serine phosphorylation of p120 catenin occurs after stimulation of cells by epidermal growth factor (EGF), colony-stimulating growth factor (CSF-1) and platelet-derived growth factor (PDGF), and correlates with transformation in cells transfected with v-Src. p120 catenin associates with the cytoplasmic domain of E-cadherin, which targets p120 catenin to cell-cell junctions, where it functions in development, morphogenesis and tumorigenesis. Additionally, p120 catenin regulates Rho GTPases, inhibiting RhoA and activating Rac and cdc42. Evidence indicates that p120 can both positively and negatively regulate adhesion, depending on expression levels, localization, association with E-cadherin/Rho GTPases, and isoform (alternatively spliced) expressed.

The present inventors also have discovered that ZC3 kinase associates with Abi-1 and Abi-2 proteins, via the SH3 domains of those proteins, in cells. Moreover, the inventors have discovered that ZC3 kinase strongly phosphorylates Abi proteins.

Thus, according to the present invention, Abi-1 and Abi-2 constitute useful ZC3 kinase substrates. Abi-1 and Abi-2 were first identified by their ability to interact with the c-Abl tyrosine, and were initially described as a suppressors of v-Abl transforming activity. Abi-1, also known as E3b1, was shown to be involved in cytoskeletal reorganization by transducing signals between Ras and Rac. The role of Abi proteins in modulating the actin cytoskeleton is further demonstrated by the fact that they localize to sites of actin polymerization at the tips of lamellipodia and filopodia.

Abi-1 and Abi-2 contain SH3 domains and polyproline motifs (PXXP) that allow them to interact with various proteins. They can interact with SH3-containing proteins through their PXXP motifs and with PXXP-containing proteins through their own SH3 domain. Indeed, the present inventors have found that the PXXP motifs in ZC3 interact with Abi-1 and Abi-2.

As used herein, the term “modulates” refers to the ability of a compound to alter kinase activity. Thus, modulation includes activation and inhibition of kinase activity. “Activation” refers to increasing the cellular activity of a kinase, whereas “inhibition” refers to decreasing the cellular activity of a kinase. Preferably, a modulator inhibits kinase activity. Modulation may result directly from interaction of a compound with a kinase molecule, or indirectly from interaction of a compound with other elements that impact kinase activity. For example, a modulator may alter the function of a kinase by increasing or decreasing the probability that a complex forms between the kinase and a natural binding partner.

The “base level of phosphorylation” refers to the level of substrate phosphorylation in a mammal before exposure to a compound that modulates the relevant kinase activity. For example, in a method for determining whether a compound modulates ZC1 kinase activity in a mammal, the base level of phosphorylation might be a measured level of p120 catenin phosphorylation prior to administration of a test compound. Similarly, in a method for determining whether a mammal is responding to a compound that modulates ZC3 kinase activity, a base level of phosphorylation might be a measured level of Abi-2 phosphorylation from prior to administration of the modulating compound. Methods for determining a base level of phosphorylation are described herein.

In comparison, a “standard level of phosphorylation” refers to the level of substrate phosphorylation in a mammal of the same species that has not been exposed to a compound that modulates the relevant kinase activity. The standard level may be determined from substrate phosphorylation in an individual mammal, but is more meaningful if determined from substrate phosphorylation in a population of mammals. By way of example, in a method for determining whether a compound modulates ZC1 kinase activity in a mammal, a standard level of phosphorylation might be the level of p120 catenin phosphorylation in another mammal of the same species or an average level of p120 catenin phosphorylation in a group of mammals of the same species. Similarly, in a method of predicting whether a mammal will respond therapeutically to a ZC3 kinase modulator, a standard level of phosphorylation might be the level of Abi-1 phosphorylation in another mammal of the same species or an average level of Abi-1 phosphorylation in a group of mammals of the same species.

Methods for measuring substrate phosphorylation are well known in the art. As the accompanying examples illustrate, one method involves isolating substrate proteins by immunoprecipitation, followed by measurement of reactivity with a labeled phospho-threonine specific antibody. A more streamlined, and preferred, method for measuring substrate phosphorylation employs labeled antibodies that specifically bind phosphorylated residues on a substrate molecule. Examples would include monoclonal antibodies or antibody fragments that specifically bind to residues on p120 catenin or Abi-1 that have been phosphorylated by ZC1 and ZC3, respectively. Such antibodies also are useful for performing in situ hybridization. Phosphorylation levels can be visually evaluated or can be quantified using well known technologies, such as ELISA, SDS PAGE, Western Blot and immunoprecipitation

According to the present invention, a mammal responds “therapeutically” when factors contributing to an abnormal condition are ameliorated to some degree. A therapeutic response can refer to one or more of the following: (a) an increase or decrease in cell proliferation, growth, and/or differentiation; (b) an increase or decrease in cell death; (c) a decrease in degeneration; (d) relief of symptoms associated with an abnormal condition; and (e) enhancing the function of an affected population of cells. Preferably, the abnormal condition is cancer, and the therapeutic response is a decrease in malignant cell proliferation and/or growth.

Methods of the present invention may be performed on any mammal, including a human, rat, mouse, dog, rabbit, pig, sheep, cow, horse, cat, primate or monkey. Preferably, the mammal is a rat, mouse or primate. Most preferably, the mammal is a human.

The inventive methods may be performed in vitro. In some embodiments, phosphorylation is measured in at least one biological tissue, such as buccal mucosa tissue, skin, hair follicles, tumor tissue or bone marrow. In other embodiments, phosphorylation is measured in at least one biological fluid, such as whole blood, peripheral blood mononuclear cells, plasma, urine or saliva.

In some embodiments, the inventive methods are performed on mammals that have cancer. The cancers may be, but are not limited to, prostate cancer, colorectal cancer, thyroid cancer, an advanced solid malignancy, pancreatic cancer, breast cancer, parotid cancer, synovial cell cancer or sarcoma, gastrointestinal stromal tumor, laryngeal cancer, testicular cancer, leiomyosarcoma, rectal cancer, gall bladder cancer, hepatocellular cancer, melanoma, ovary cancer, lung cancer, colon cancer, renal cell carcinoma, sarcoma, retropero sarcoma, pelvis sarcoma, uterine cancer, pelvic angiosarcoma, pleural mesothelioma, neuroendocrine cancer, bronchial adenocarcinoma, head and neck cancer or thymic cancer.

“Exposing,” “administer,” and “administration” refer to the delivery of a compound to a mammal. Suitable routes of administration include oral, rectal, transmucosal or intestinal administration or intramuscular, subcutaneous, intramedullary, intrathecal, direct intraventricular, intravenous, intravitreal, intraperitoneal, intranasal, or intraocular injections. The preferred routes of administration are oral and parenteral.

Administration of a compound may occur in any of numerous forms known in the art. Proper formulation is dependent upon the route of administration chosen. The compounds may be administered alone or as part of a pharmaceutical composition. A “pharmaceutical composition” refers to a mixture of at least one compound with physiologically/pharmaceutically acceptable carrier(s) and/or excipient(s). The purpose of a pharmaceutical composition is to facilitate administration of a compound to an organism.

“Physiologically/pharmaceutically acceptable carrier” refers to a carrier or diluent that does not cause significant irritation to an organism and does not abrogate the biological activity and properties of the administered compound.

“Pharmaceutically acceptable excipient” refers to an inert substance added to a pharmaceutical composition to further facilitate administration of a compound. Examples include calcium carbonate, calcium phosphate, various sugars and types of starch, cellulose derivatives, gelatin, vegetable oils, polyethylene glycols and the like.

Determination of a proper dose is well within the capability of those skilled in the art, especially in light of the detailed disclosure provided herein. For any compound used in the methods of the invention, the dose can be estimated initially from cell culture assays. Then, the dosage can be formulated for use in animal models so as to achieve a circulating concentration range that includes the IC₅₀ as determined in cell culture. Such information can be used to more accurately determine useful doses in humans.

Methods of the invention may be used to individually adjust dosage amounts and intervals, to provide plasma levels of the active species that are sufficient to maintain the kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration.

Methods of the invention are performed so that the step where a mammal is exposed to a compound includes administration of at least one dose of the compound, or at least two doses, or at least 5 doses or at least 10 doses, up to at least 55 or 56 doses. In certain embodiments, these doses are administered during a period of 4 hours, 6 hours, or 24 hours to about 100 days. In further embodiments, the doses are administered over a period of 24 hours, 2 days, or 28 days. In other embodiments, two doses are administered per every 24 hours, and in other embodiments, the doses are administered about every 12 hours. It will be understood by those of skill in the art that the administration of a compound, according to the exposure steps of the methods, can be varied to suit individual needs of the mammal being treated, the compound being administered, the method of administration and the disease being treated. For example, in a typical dosing regimen, the patient receives one dose per day of the compound, for a number of days, such as about 28 or about 56 days. In other dosing regimens, the compound is administered about once per day, twice per week, or once per week.

The measurement of substrate phosphorylation, following the exposure step in the methods, can be performed on a sample from the mammal taken about 4 or 6 hours following the first dose (exposure) of the mammal to the compound. In other embodiments, this measurement is performed on a sample taken 12 hours, 1 day, 2 days, up to about 100 days, after the first dose (exposure) of the mammal to the compound. In other embodiments, the phosphorylation measurements are taken from samples from the mammals 4 or 6 hours after the first dose of compound or 24 hours after the first dose of compound, or 15 or 28 days after the first dose of compound. Typically, dosing of compound will be periodic between the first and last dose of compound that precedes the sample taken for measurement of phosphorylation. For example, the compound is administered once a day, every day for 28 days. Typically, the mammal sample taken (for measurement of phosphorylation) will be taken shortly following the most recent dose of compound, for example within 24 of the most recent dose of compound.

The difference in the level of phosphorylation measured in the inventive methods (e.g., “base” level vs. post-exposure or “standard” vs. tumor) may be an increase or decrease of at least about 10%, 15%, 20%, 25%, 30%, 35%, 50%, 75% or 100%. Preferably, it is an increase or decrease of at least about 25%. In some embodiments, the difference may be an increase or decrease of at least 2-, 3- 5-, 10-, 15-, or 20- fold. In still other embodiments, the difference in the level of phosphorylation is an increase or decrease of at least 1.1-, 1.2-, 1.3-, 1.4-, 1.5-, 1.6-, 1.7-, 1.8-, 1.9-, 2.0-, 2.5-, 3.0-, 3.5-, 4.0-, 5.0-, 7.5-, 10.0-, 15.0- or 20.0- fold.

In addition to the above-described methods, the invention includes a kit comprising: (a) a phospho-threonine-specific antibody or antibody fragment, and (b) instructions for performing one of the methods described herein.

Reference to the following illustrative examples will help to provide a more complete understanding of the invention.

### EXAMPLE 1

### Endogenous ZC1 co-localizes with p120 catenin at cell-cell junctions

This example demonstrates that ZC1 kinase co-localizes with p120 catenin at cell-cell junctions.

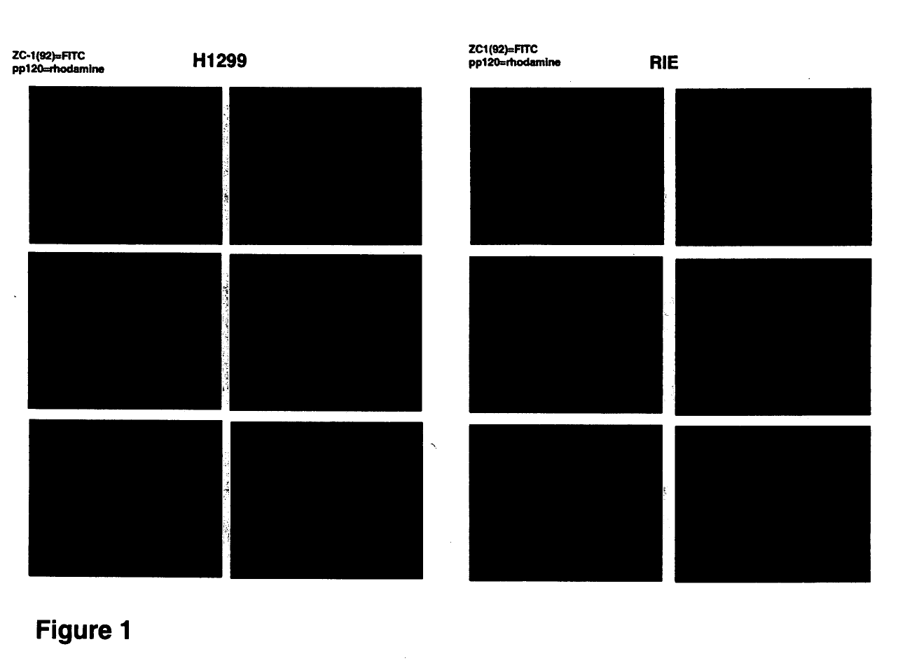

ZC1 antibodies were raised and used to determine where ZC1 is localized in cells. In H1299 cells, as well as in rat intestinal epithelial cells (RIE-1), anti-ZC1 antibodies showed a punctate cytoplasmic staining, with a significant amount of the staining also at points of cell-cell contact (FIG. 1, top panels). In cells that do not form adherens junctions, ZC1 staining showed the punctate, cytoplasmic staining pattern only (data not shown). The nuclear staining observed with this antibody, especially prominent in the RIE-I cells was not observed with other ZC1 antisera, nor with anti-myc in cells expressing myc tagged-ZC1, so we do not believe that this represents a real population of nuclear ZC1, but rather background staining. The ZC1 localized at cell junctions as well as in the cytoplasm granular structures was observed with two other independently isolated antisera (data not shown). H1299 and RIE-1 were also stained for p120 catenin as a marker for adherens junctions (FIG. 1, middle panels). As expected, p120 was localized to cell-cell junctions, as well as in the cytoplasm. When the two images were merged (FIG. 1, lower panels), the p120 and ZC1 at cell-cell junctions overlapped. ZC1 IF was performed on cells over-expressing ZC1; the over-expressed kinase gave strong staining throughout the cell, precluding identification of specific localization.

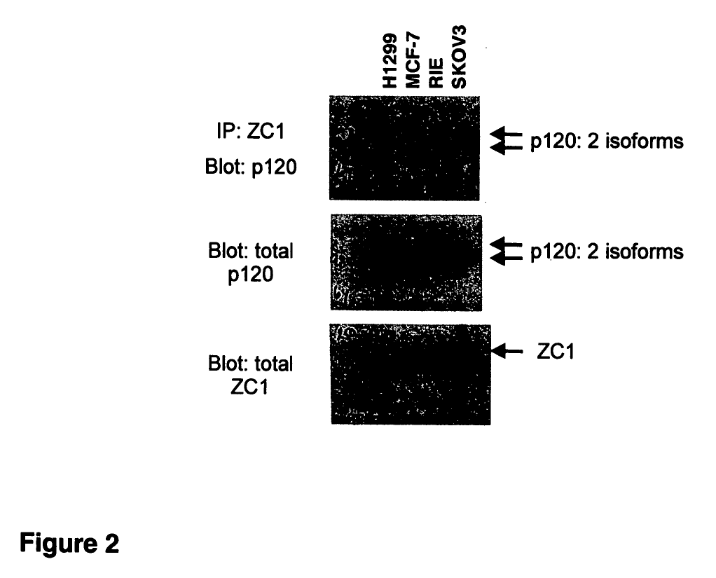

To corroborate the conclusion that a portion of the cellular ZC1 is localized to adheren junctions, we demonstrated that ZC1 and p120 associate in cell lysates. Confluent plates of each of four cell lines, H1299, MCF-7, RIE-1, and SKOV-3 were lysed in detergent, and the cellular supernatants were subjected to immunoprecipitation with anti-ZC1 antisera. When immunoprecipitates were analyzed in Western blots probing for endogenous p120, two bands corresponding to two isoforms of p120 were observed to co-purify with ZC1 in the immunoprecipitate (FIG. 2, top panel). Associated p120 was most apparent in H1299 and RIE-I cells, which form adherens junctions and contain a higher level of endogenous p120 that the other 2 cell types that did not form junctions (MCF-7 and SKOV-3). This experiment was repeated under harsher extraction conditions (RIPA buffer), and the co-purification of p120 in the ZC1 immunoprecipitates was still apparent (data not shown). To test for the specificity of this co-immunoprecipitation, we over-expressed both p120 and ZC1 in H1299 cells in different combinations and repeated this co-immunoprecipitation experiment. No significant p120 was retained on the beads in cells that did not have ZC1 co-transfected along with the p120. In this experiment, far less lysate than for analysis of co-immunoprecipitation with the endogenous ZC1 was used so the amount of endogenous ZC1 in H1299 becomes insignificant. Endogenous p120, however, being more abundant in this cell type, can also be seen to co-immunoprecipitate with the over-expressed ZC1 where it was added. Note that the apparent molecular weight of the exogenous p120 was different due to the presence of an HA-tag.

### EXAMPLE 2

### ZC1 phosphorylates p120 catenin in vitro

This example demonstrates that ZC1 kinase phosphorylates p120 catenin in vitro.

Co-transfection with a plasmid encoding p120, with and without a plasmid encoding active full-length ZC1, into 293T cells with lipofectamine (Invitrogen) was performed. After 48 hours, transfected cells were harvested in lysis buffer (50 mM HEPES (pH 7.0), 150 mM NaCl, 1.5 mM MgCl₂, 1 mM EGTA, 10% glycerol, 1% Triton, 10 mM pyrophosphate, 1 mM Na₃VO₅, 1 mM DTT, 0.1 mg/ml of 4-(2-aminoethyl)benzenesulfonyl fluoride, 2-10 mg/ml each of aproptonin, pepstatin, leupeptin and E-64), incubated on a rocker for 30 minutes and clarified by centrifugation. Supernatants were precleared with protein A sepharose and immunoprecipitated with anti-p120 antibody (and protein A sepharose) for 2 hours at 4° C. After incubation, immune complexes were washed four times in lysis buffer and then 2 times in kinase assay buffer. Washed complexes were incubated with kinase assay buffer containing 100 micromolar ³²P-ATP with or without 4 micrograms purified GST-ZC1 kinase domain for 30 minutes at 30° C. Reactions were stopped with Laemmli SDS sample buffer and analyzed by SDS-PAGE, stained with GelCoat coomassie stain (Bio-Rad) and analyzed by autoradiography.

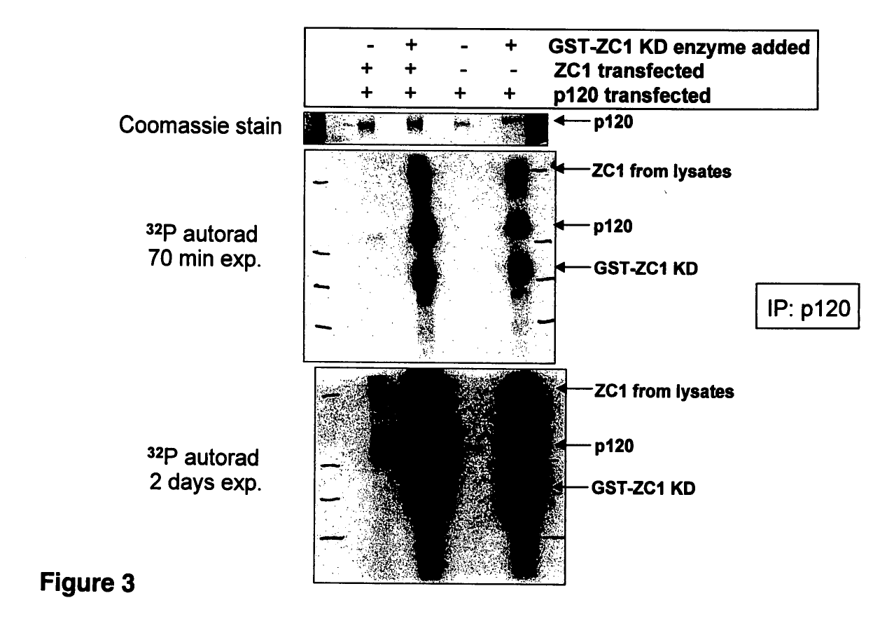

Results are shown in FIG. 3. ZC1 was found to phosphorylate p120 in vitro. When p120 was isolated from cells by immunoprecipitation with anti-p120 antibody, added purified recombinant ZC1 enzyme induced phosphorylation of p120 as shown in the lower 2 panels. In addition, p120 immunoprecipitated from cells co-expressing ZC1 also showed phosphorylation of p120 in vitro by associated ZC1, which co-immunoprecipitated out of the cell extract. The fact that phosphorylation by added ZC1 enzyme caused a gel mobility shift in the stained p120 protein band (shown in top panel) indicated that p120 was quantitatively phosphorylated by ZC1 in vitro, and indicated that it served as a very efficient substrate for ZC1 kinase.

### EXAMPLE 3

### ZC1 phosphorylates p120 catenin in vivo

This example demonstrates that ZC1 kinase phosphorylates p120 catenin in vivo.

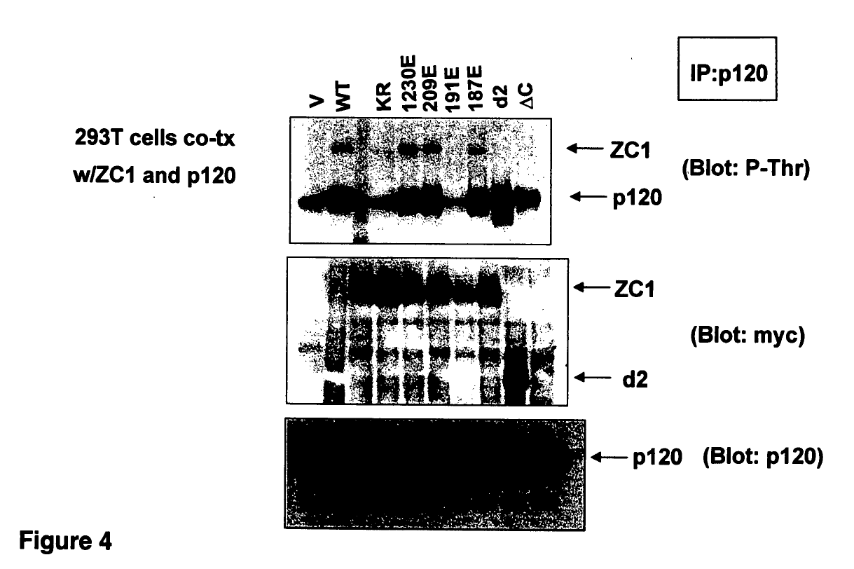

Co-transfection with a plasmid encoding p120, with and without a plasmid encoding different forms of ZC1, all tagged with myc (WT=wild type; KR=kinase-inactive; 1230E=T1230E phosphorylation site mutant; 209E=T209E phosphorylation site mutant; 191E=T191E inactive phosphorylation site mutant; 187E=T187E phosphorylation site mutant; d2 and DC both contain deletion mutants of ZC1 that retain the kinase domain), into 293T cells with lipofectamine (Invitrogen) was performed. After 48 hours, transfected cells were harvested in lysis buffer (50 mM HEPES (pH 7.0), 150 mM NaCl, 1.5 mM MgCl₂, 1 mM EGTA, 10% glycerol, 1% Triton, 10 mM pyrophosphate, 1 mM Na₃VO₅, 1 mM DTT, 0.1 mg/ml of 4-(2-aminoethyl)benzenesulfonyl fluoride, 2-10 mg/ml each of aproptonin, pepstatin, leupeptin and E-64), incubated on a rocker for 30 minutes and clarified by centrifugation. Supernantants were precleared with protein A sepharose, and immunoprecipitated with anti-p120 antibody (and protein A sepharose) for 2 hours at 4° C. After incubation, immune complexes were washed four times in lysis buffer and subjected to SDS-PAGE in parallel gels. Western blots of those gels were probed with anti pThr (Cell Signaling), anti-myc for ZC1 ((E10 in house hybridoma sup), and anti-p120 (Becton-Dickenson). Results are shown in FIG. 4.

To show that p120 was also a substrate for ZC1 in the cell, the phosphorylation state of p120 in the cell was examined with a phospho-threonine antibody. A plasmid encoding p120 protein was co-transfected into 293T cells, alone and in combination with 8 different forms of ZC1, including both activate and inactive forms of the enzyme. When immunoprecipitated from cell extracts, p120 reactivity with the phospho-threonine antibody increased in those extracts prepared from cell also transfected with active forms of ZC1 (FIG. 4, top panel, lane 2,5,6, and 8 from the left), as compared to cells transfected with p120 alone (first lane on the left) or cell co-expressing inactive forms of ZC1 (lanes 4 and 7). Truncated forms of ZC1 were less effective at increasing p120 reactivity with pThr antibody than full length forms (lanes 9 and 10 from the left), but still did so significantly more than inactive forms of the enzyme. In addition, co-transfected ZC1 protein was found associated with the isolated p120, corroborating the data from Example 1 (anti-ZC1 blot shown in middle panel). Total levels of p120 protein in the immune-complex are shown by anti-p120 western blot (bottom panel).

### EXAMPLE 4

### ZC3 kinase selectively interacts with Abi-1 and Abi-2

This example demonstrates that ZC3 kinase selectively interacts with Abi-1 and Abi-2 proteins. More particularly, the linker region of ZC3 (aa 672-1032 of ZC3 allele), which contains several PXXP motifs, interacts with SH3 domains on Abi-1 and Abi-2.

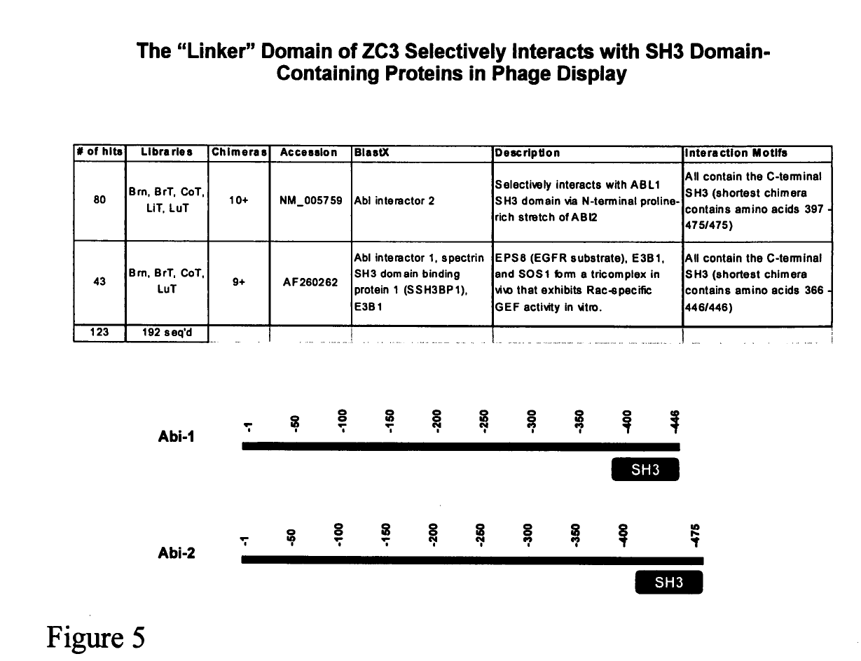

A pGEX expression construct containing the linker domain of ZC3 was constructed. The linker domain (amino acids 672-1032 of ZC3 allele) was arbitrarily defined as the region that lies between the NCK-interaction domain and the CNH domain. The resulting GST-ZC3-linker protein was immobilized on glutathione-Sepharose resin and used to pan several phage display libraries (Novagen T7Select10-3- normal brain, breast tumor, colon tumor, liver tumor and lung tumor cDNA). After three rounds of enrichment, 32-40 individual plaques from each library were sequenced. Results are summarized in FIG. 5. Of 192 inserts sequenced, 124 contained an in-frame SH3 domain: 80 were Abi-2 chimeras; 43 were Abi-1 chimeras; 1 was a Cbl-associated protein chimera. Thus, the SH3 domains of Abi-1 and Abi-2 directly interact with ZC3 in phage display.

### EXAMPLE 5

### ZC3 kinase selectively interacts with Abi-1 and Abi-2 intracellularly

This example demonstrates that ZC3 kinase selectively interacts with Abi-1 and Abi-2 proteins intracellularly.

COS7 cells were transfected with the following plasmids: ZC3: pcDNA-ZC3, ZC3B: pcDNA-ZC3B, Abi1: pCMV-Flag-Abi1, Abi2B: pCGN HA-Abi2B. After 48 hours, cells were lysed in HNTG buffer (50 mM HEPES pH7.5, 10% glycerol, 150 mM NaCl, 1% Triton X-100, 1.5 mM MgCl₂, 1 mM EGTA) containing protease inhibitors. After measuring protein concentration using the BCA assay (Pierce), 200 ug of cell lysates were subjected to immunoprecipitation with either an antibody against ZC3 (292B), an anti-Flag antibody (Sigma) to bring down Abi1, or an anti-HA antibody (12CA5) to bring down Abi2B. Protein A and Protein G sepharose beads (Amersham Biosciences) were used to bring down the immune-complexes, which were washed four times in HNTG buffer before being processed for SDS-PAGE. Several gels were run and probed with the different antibodies. Total lysates were also subjected to western blotting to confirm that the various constructs transfected into the cells were correctly expressed (two lower blots on each panel). The anti-Abi antibody, #5421, used for western blotting was described in Dai, Quackenbush et al. 1998.

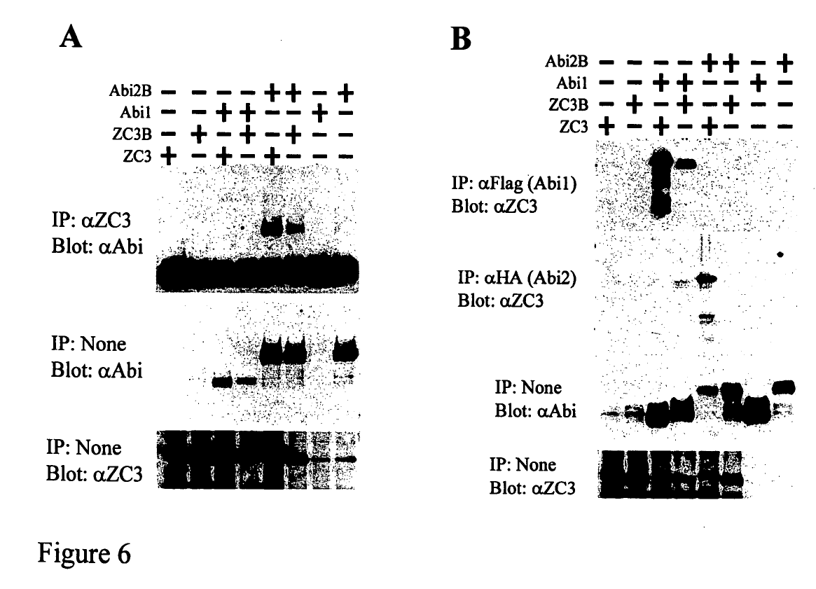

FIG. 6 depicts the results, which show that ZC3 interacts with Abi-1 and Abi-2B in cells. When ZC3 or ZC3B, a splice variant of ZC3, was co-transfected with Abi-1 or Abi-2B into COS-7 cells, ZC3 co-immunoprecipitated with Abi proteins, and conversely Abi proteins co-immunoprecipitated with ZC3. Panel A shows that Abi-2B can co-immunoprecipitate with ZC3 and ZC3B when they are co-expressed in cells. Abi-1 is not visible because it runs too close to the IgG heavy chain band (thick band across the blot). Panel B shows that both ZC3 and ZC3B can co-immunoprecipitate with Abi-1 (top blot), and that ZC3 can co-immunoprecipitate with Abi-2B (second blot from the top).

### EXAMPLE 6

### ZC3 phosphorylates Abi-1 in vitro

This example shows that ZC3 kinase phosphorylates Abi-1.

Abi-1 was immunoprecipitated from 293T cells using an anti-Flag antibody. Immunoprecipitations performed on lysates from 293T cells transfected with pcDNA3, and without any lysate were used as negative controls. The immunoprecipitated protein bound to Protein A Sepharose beads was subjected to a kinase assay (20 mM Tris HCl pH 7.4, 200 mM NaCl, 10 mM MgCl₂, 1 mM DTT, 100 uM ATP) plus 5 uCi of [y³²P]ATP per 50 ul reaction. The kinase domain of ZC3 fused to GST (190 ng of protein) was added to the samples. The kinase reaction was allowed to proceed for 20 min at 30° C., with agitation, and was stopped by adding SDS sample buffer and incubating 3 min at 95° C. GST-ZC3KD alone and plus Myelin Basic Protein (MBP, 5 ug) samples were also included as controls. The samples were processed for SDS-PAGE (4-20% gradient gel, Invitrogen), the gel was stained (Gel Code, Pierce), dried and autoradiographed.

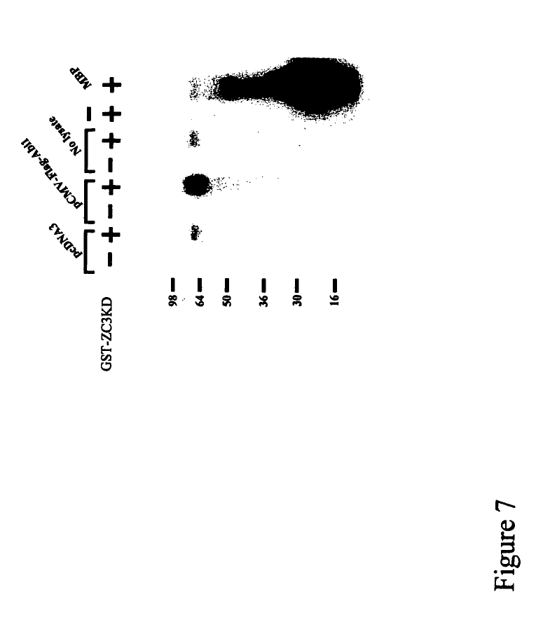

FIG. 7 depicts the results. A strongly phosphorylated band corresponding to Abi-1 appears in the sample containing immunoprecipitated Abi-1 and GST-ZC3KD. The weaker band in the other lanes corresponds to GST-ZC3KD itself; its expected size is 63 KD. MBP is known to be a good in vitro substrate for ZC3 and is indeed strongly phosphorylated.

### EXAMPLE 7

### HGK (ZC1) functions in tumor cell growth in vitro and in vivo, and can regulate cell-cell and cell-substrate adhesion

This example shows that over-expression of HGK (ZC1) promotes anchorage-independent growth, that HGK siRNA reduces anchorage-independent growth, that HGK promotes tumor growth in vivo, and that HGK promotes cell rounding and loss of cell-cell contacts.

### MATERIALS AND METHODS

Cell lines and transfection methods

H1299, HeLa, and A549 human cell lines and other tumor cell lines were originally obtained from the ATCC (Manassas, Va.). All cell lines were grown in DMEM medium (Invitrogen, Carlsbad, Calif.), supplemented with 10% fetal bovine serum (FBS, Invitrogen) at 37° C. with 5% CO₂. Construction of pcDNA3 plasmids encoding the wild type and K54R mutant can be found in Wright et al, 003. To express HGK wild type (WT) and inactive mutant (KR), H1299 cells were transfected with 5 ug pcDNA3/HGK construct using Lipofectamine 2000 (Invitrogen). Stable pools and clonal populations of transfected cells were selected using 500 ug/ml G418 (Invitrogen) over the course of one to two weeks post-transfection. Transfectants over-expressing either wild-type HGK (WT), kinase inactive HGK (KR) or vector alone (V) were cultured in an identical manner as the parental cells from which they were derived.

RNA interference

Oligos were prepared 2′-deprotected, annealed, purified and lyophilized. They were generated with dTdT/dTdT overhangs on the sense and antisense strands. Two siRNA duplexes against the HGK ORF were prepared (sense strand shown):

| | |
|---|---|
| duplex 4: 5′AAGAAGAGGAGGAAGUGCCUG | (SEQ ID NO:1) |
| duplex 8: 5′AACACAUAUGGAAGGAUCACC | (SEQ ID NO:2) |

The duplexes were resuspended in sterile water at a stock concentration of 40 μM. A scrambled siRNA sequence also was prepared:

| | |
|---|---|
| 5′GCGCGCTTTGTAGGATTCG | (SEQ ID NO:3) |

The scrambled target sequence is not present in mammalian cells as determined by BLAST search at NCBI: www.ncbi.nim.nih.gov/BLAST/. A LaminAC siRNA sequence also was prepared:

| | |
|---|---|
| 5′CTGGACTTCCAGAAGAACA. | (SEQ ID NO:4) |

Cell lines (4.5×10⁴ cells) were seeded in 12-well dishes and allowed to adhere for six hours. Cell lines were transfected with either varying concentrations of HGK siRNA reagents, scrambled siRNA or siRNA reagents against other genes using Oligofectamine (Invitrogen) in growth medium without antibiotics as follows: 5 μl of oligofectamine and 20 μl of Opti-MEM were mixed and incubated for 10 minutes at room temperature, then mixed with the siRNA reagents (19 μl volume or less) which had been diluted with 100 μl of Opti-MEM Growth medium. The oligofectamine/siRNA suspension was incubated for 25 minutes at room temperature. The medium in each well was replaced by 0.8 ml of fresh medium containing FBS. The lipofectamine-siRNA complexes were added drop wise. Seventy-two hours post-transfection a second round of transfection with siRNA was performed. Cells were allowed to recover for an additional 24 hours. siRNA transfected cells (1.0×10³, 5×10³ cells) were plated in 0.25% Bactoagar (Gibco-BRL)-ISCOVE's-10% FBS on top of a 0.75% Bactoagar-ISCOVE's-10% FBS base layer. For each cell type, treatments with or without siRNAs were performed in two 60 mm dishes. After seven days, plates were fed with 0.75% Bactoagar/ISCOVE's-10% FBS. Soft agar plates were stained with Neutral Red (Invitrogen) after 14 days and scanned and quantified with Prolmage Software.

Cell growth assays

Monolayer growth. Proliferation assays were performed as follows: 1×10⁵ cells were plated in each well of a 6-well tissue culture plate. After 24 hours, growth medium was removed and replaced with DMEM containing 1% or 10% fetal calf serum (Invitrogen). Cells were removed from plates with trypsin and counted using a hemocytometer on days 1, 3 and 5 following.

Soft agar assay. This assay was performed to determine the ability of cells to grow in an anchorage-independent manner. Stock soft agar solution was prepared by autoclaving 1.6% bacto-agar (Difco, Becton Dickenson, Franklin Lakes, N.J.) in H₂O. Stock agar was diluted in Iscove's Modified Dulbecco's Medium (IMEM, Invitrogen) to various concentrations. 6 cm² plastic tissue culture dishes were coated with 2.5 ml 0.8% agar/FBS (5% or 10%), allowing for polymerization before a middle layer of 1×10⁴ cells in a mixture of 0.4% agar (plus 5% or 10% FBS, 2.5 ml) was added. After polymerization of the middle layer, a final layer of 2.5 ml of 0.8% agar/FBS was layered on top of the cells. Assays were carried out for 4 weeks, with weekly “feedings” of 0.8% agar/FBS/IMEM. Colonies were observed and photographed using a Zeiss Axiovert 100 inverted microscope. Plates were fixed in 10% acetic acid+10% MeOH and stained with 0.1 % crystal violet in 20% MeOH. Colony number was tabulated by scanning the plates and counting manually.

In vivo cell inoculation

Cells were routinely prepared for inoculation by centrifugation and washing twice, followed by resuspension at 5 million cells/100 μl 1× PBS (cells were determined to be at least 90% viable). Cells were then injected subcutaneously into the right dorsal flank of 6-8 week old intact male NCr nu/nu mice (Charles River Laboratories, Wilmington, Mass.). Beginning approximately 2 weeks post-injection, xenografted tumors were measured weekly and monitored for growth and/or necrosis. Animals were sacrificed by CO₂ asphyxiation as tumors reached 2 cm³ or before, if they showed any sign of discomfort, paralysis or emaciation. Tumor xenografts were excised and immediately snap frozen in liquid nitrogen, ground into a fine powder and stored for use at −80° C. When needed, powder was lysed in HGK lysis buffer and assayed by western blotting.

Immunofluorescence

Cells were plated on glass cover slips, grown to approximately 70% confluence, fixed with 4% para-formaldehyde (EMS, Ft. Washington, Pa.), permeabilized with 0.2% Triton-X100/PBS and nonspecific sites blocked by incubation with 3% FBS. Incubation with Texas red-conjugated phalloidin (dilution of 1:100) for one hour was followed by counterstaining with 10 ng/ml DAPI (Pierce). Cover slips were washed, mounted onto microscope slides and examined using the Nikon Microphoto FXA microscope.

### EXAMPLE 7A

### HGK over-expression promotes anchorage-independent growth

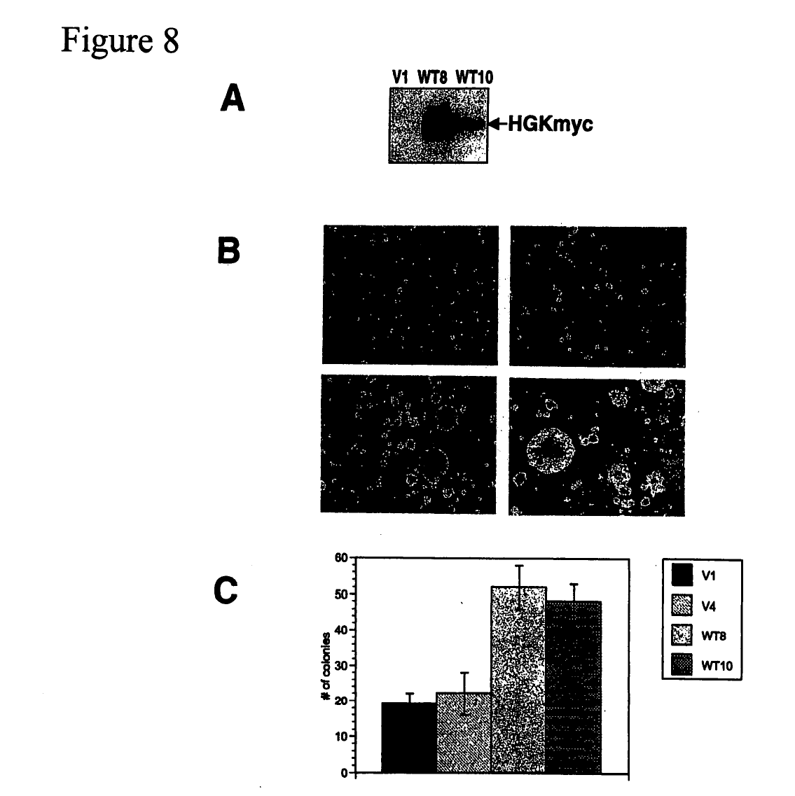

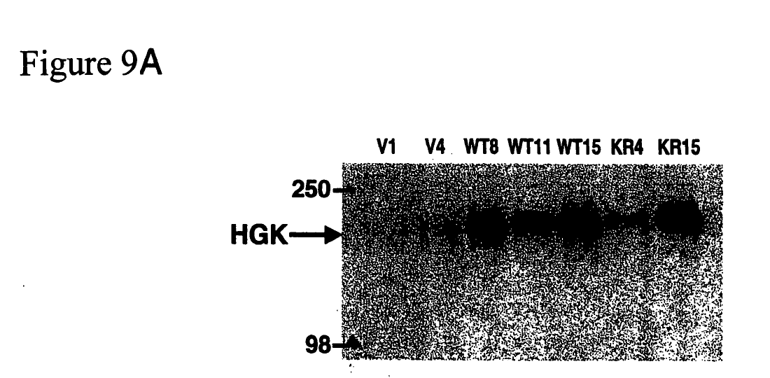

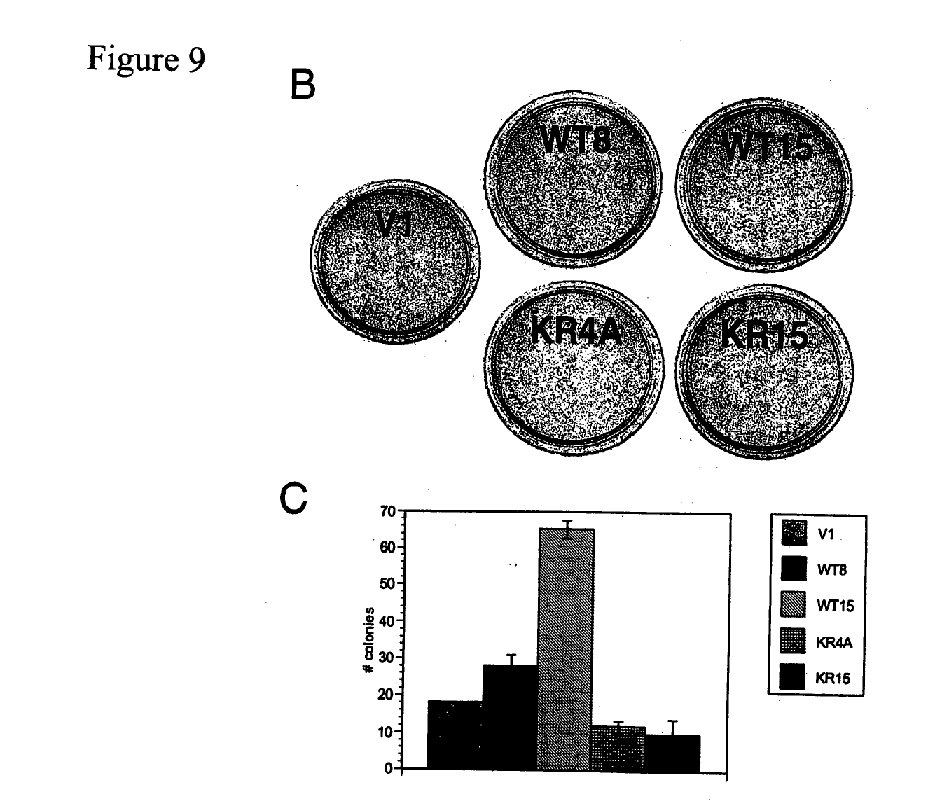

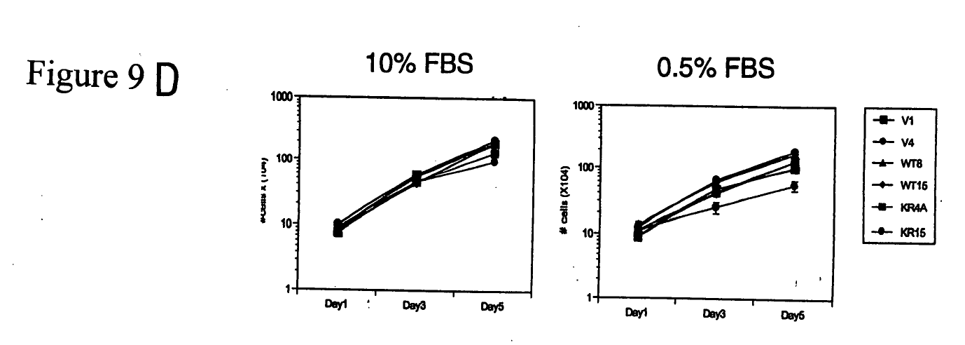

H1299 is a lung carcinoma cell line that shows slow growth in soft agar. A plasmid encoding wild type HGK, kinase-inactive HGK, or an empty vector were transfected into H1299 cells. G418-resistant clones were isolated expressing active or inactive HGK or vector alone (FIGS. 8A and 9A). The ability of these H1299 clones to grow in soft agar was assessed. In the fist study, two clones, each expressing vector alone or active HGK, were compared (FIG. 8). Both clones over-expressing HGK were found to grow faster than the two vector control clones, giving more than twice the number of colonies after two weeks of growth (FIG. 8C). In addition to giving a greater number of colonies, the average size of the colonies in the cells over-expressing wild type HGK was significantly greater (FIG. 8B).

HGK is a large kinase with multiple domains in addition to the catalytic domain. To determine if the increase in anchorage-independent cell growth was linked to the kinase activity, we did a second study comparing clones over-expressing the inactive and inactive forms of HGK, along with vector controls. FIG. 9A shows the relative expression level of HGK used in this study. As these Western blots were probed with an antisera that detects the endogenous HGK, the level of endogenous HGK can also assessed. In most all clones, HGK is over-expressed at least 10-fold over the endogenous protein levels (FIG. 9A). When compared for growth in soft agar, again we saw that both clones expressing wild type HGK had a greater number of colonies. While the net increase in colony number with the WT8 clone used in the first experiment did not show as large an increase in colony number in this second experiment, the increase in colony size was apparent (FIG. 9B). In contrast, the clones expressing the kinase inactive (KR) mutant HGK showed lower colony numbers than the vector control clones (FIG. 9B and C).

These same clones were analyzed in parallel for growth in monolayer culture in both high and low serum (FIG. 9D). In high serum, all clones appeared to have very similar growth rates. In low serum, again, the growth rates were similar, with the exception of the clone KR15, expressing the highest level of inactive HGK, which showed a slower growth rate. In contrast to the growth phenotype seen in soft agar, no growth advantage in monolayer culture was apparent with over-expression of the active wild type form of HGK, even in low serum. A similar study was done with another lung cell line, CaLu6. In this case, stable pools were analyzed. As with the H1299 cells, a potentiation of growth in soft agar was observed with expression of active HGK in CaLu6 cells.

### EXAMPLE 7B

### HGK siRNA reduces anchorage-independent growth

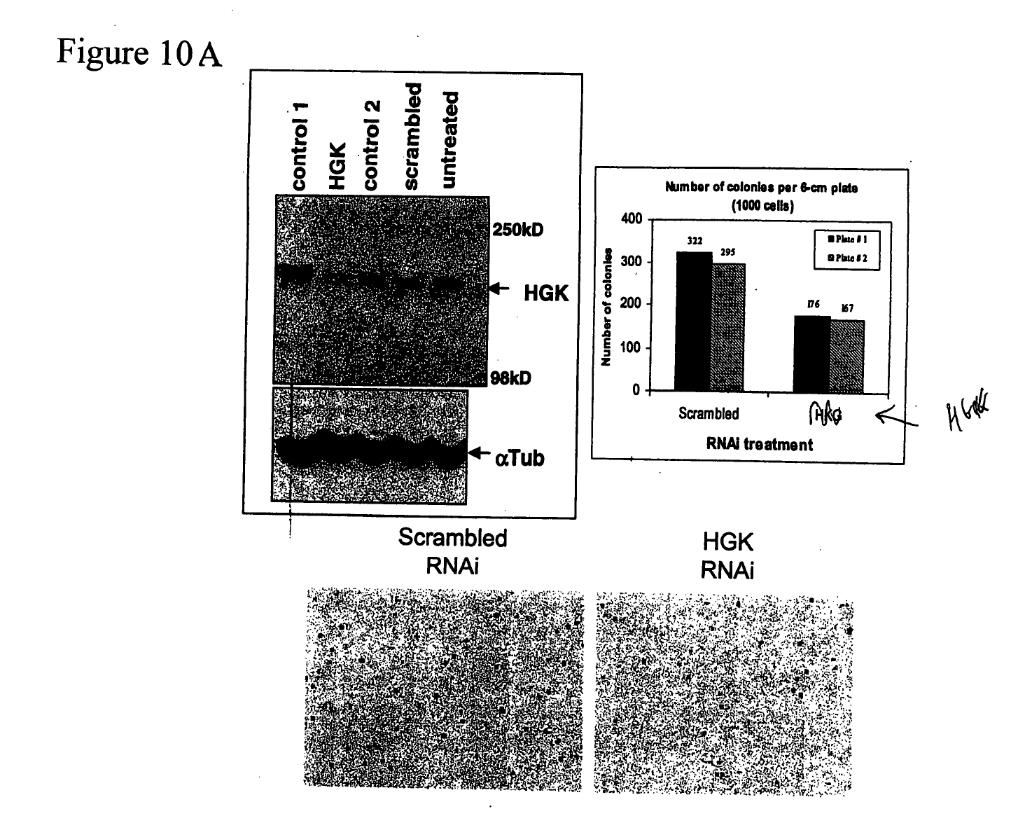

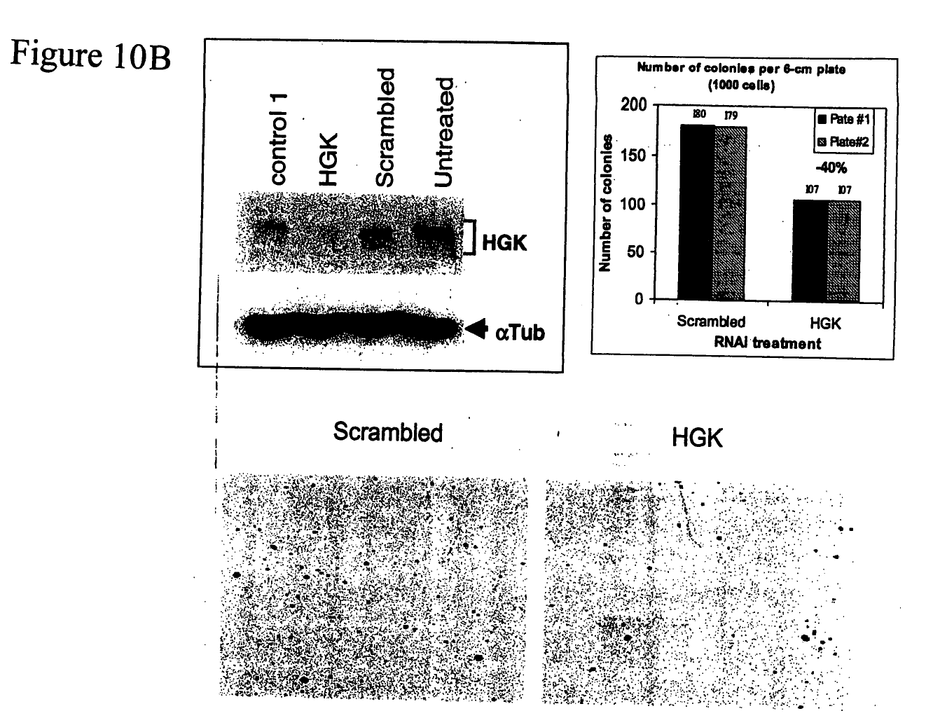

To demonstrate that HGK was important for anchorage-independent growth using another approach, we screened cell lines for the ability to significantly lower the endogenous HGK levels in tumor cells using RNA interference (siRNA). One cell type where we succeeded in lowering HGK levels was in HeLa cervical carcinoma cells. As compared with the control oligos, scrambled HGK oligos, or untreated cells, we were able to observe a reduction in the level of HGK protein (FIG. 10A). We investigated the effect of HGK siRNA versus a scrambled siRNA control in HeLa cells on anchorage independent growth (FIG. 10A). Cells were transfected twice, with a 48-hour interval. The day following the second transfection, cells were removed from plates with trypsin, counted and plated in soft agar in media containing 10% serum. Numbers of colonies formed were scored after 28 days growth. The HGK duplex treatment reduced the number of colonies formed in soft agar by 44 percent. In parallel, the growth rate of the two transfected cell populations was tested in standard monolayer growth and no difference in growth rate was observed (data not shown). We also were able to reduce endogenous levels of HGK in A549 lung carcinoma cells (FIG. 10B). Using the same transfection and plating schedule as with the HeLa cells, we again compared the anchorage-independent growth of the two transfected cell populations. In A549 cells, we were able to reduce the number of colonies by 40 percent. These data suggest the HGK kinase is important for growth of these two tumor cell lines in soft agar.

HGK kinase can promote tumor growth in vivo

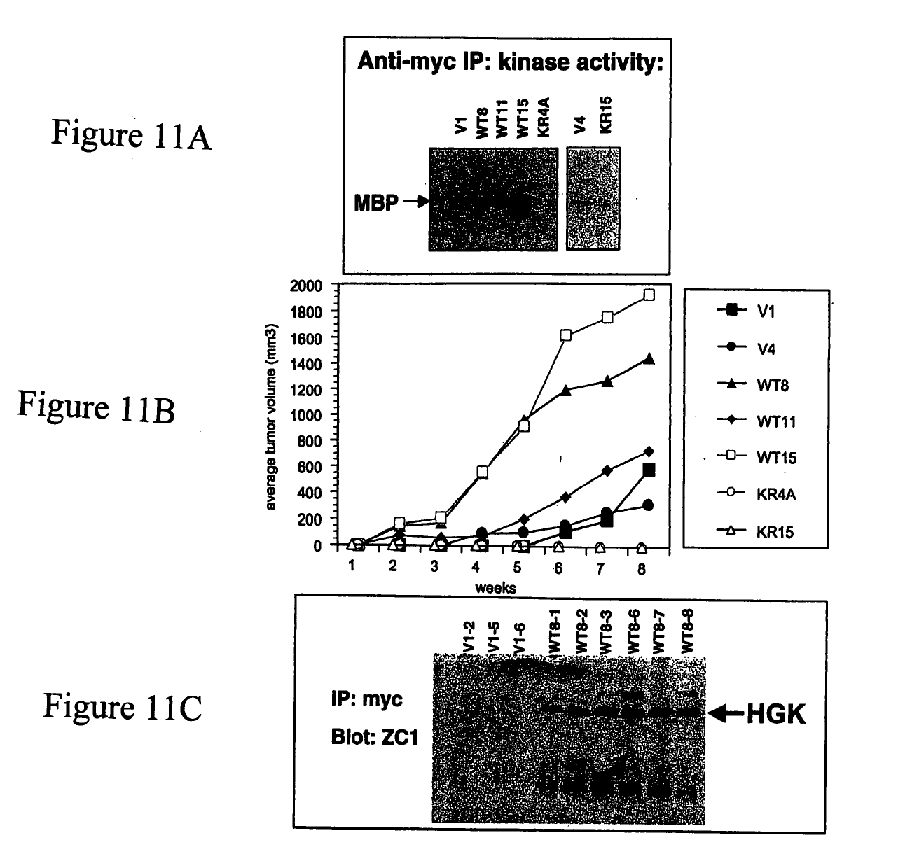

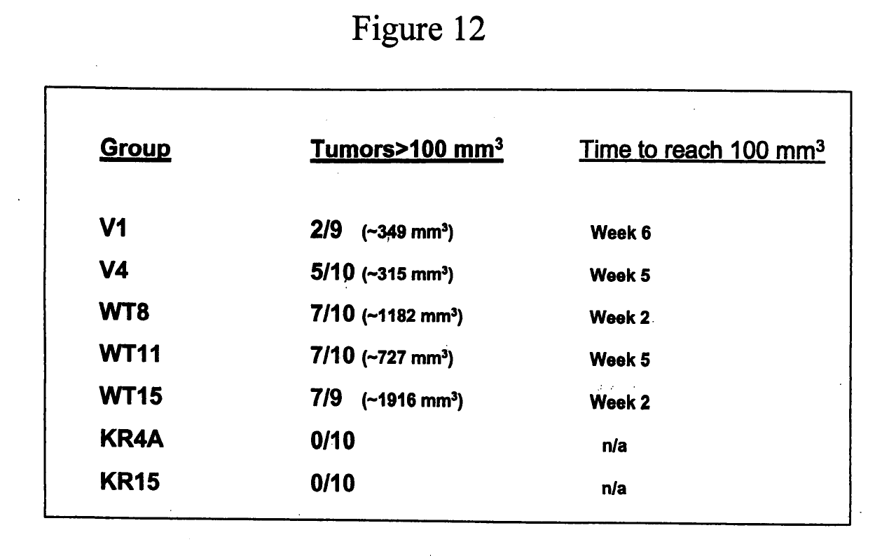

To demonstrate that over-expression of active and inactive forms of HGK can also affect H1299 tumor cell growth in vivo, we analyzed the same set of clones tested for in vitro growth in FIG. 9 for growth as subcutaneous xenograft tumors in athymic mice. Each of the 6 clones was expanded and injected into 10 mice. At the time of injection, lysates were made and tested for the relative level of HGK kinase activity introduced by performing anti-myc tag immunoprecipitations, followed by in vitro kinase assays (FIG. 11A). The relative expression level of HGK in these same clones is shown in FIG. 9A. Clone WT15 showed the highest activity, followed by WT8, and the clone showing the lowest activity was WT11. The KR clones added no HGK activity as expected. Tumor volume was measured weekly post-injection. The WTI5 and WT8 mice formed measurable tumors after only 2 weeks (>100 mm³) (FIG. 12). The two vector groups of mice, as well as WT11 mice formed tumors after 5 to 6 weeks, and the two groups of KR mice never formed tumors at all in 20/20 mice. The average tumor volume for each group of mice was calculated for each week and is shown in a graph of tumor volume vs. time in FIG. 11B. The tumor incidence for each group, as well as the average tumor volume measured at 8 weeks are also shown in FIG. 12. The control groups of mice injected with vector transfected cells showed slow tumor growth, with a relatively long latency. All 3 WT clones showed more robust tumor growth, with the WT15 and WT8 clones showing significantly faster tumor growth with a latency of less than half the time. The relative tumorigenicity of each cell line correlated with the level of HGK kinase activity. As the clones expressing the inactive HGK KR mutant showed no tumor growth, perhaps this mutant can act in a dominant-negative to block the modest growth of H1299 tumor cells in this system. The continued expression of introduced myc-tagged HGK was verified in lysates prepared from excised tumors (FIG. 11C).

HGK promotes cell rounding and loss of cell-cell contacts

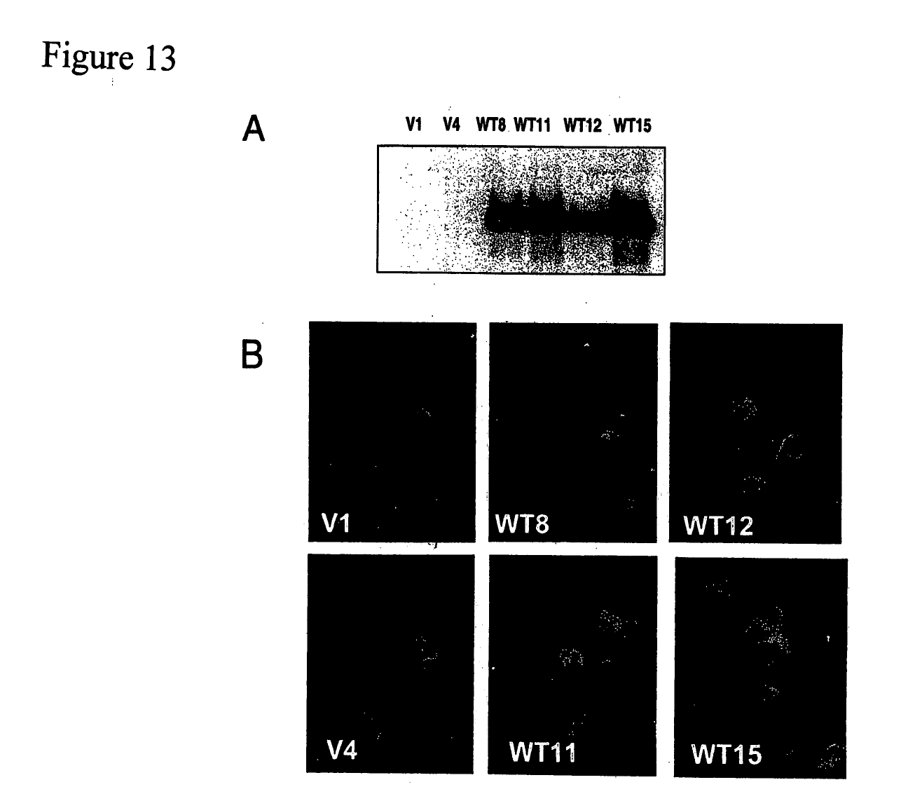

Immunofluorescence with anti-HGK antisera from two vector clones and four wild type HGK-expressing H1299 clones are shown in FIG. 13B. The morphology of the stable clones expressing wild type HGK was noticeably different from the vector clones. The vector clones grow as islands with good cell-cell contact, while the wild type clones grow separated from one another, and are less spread out on the substrate. The K54R HGK-expressing clones looked very similar to the vector clones.

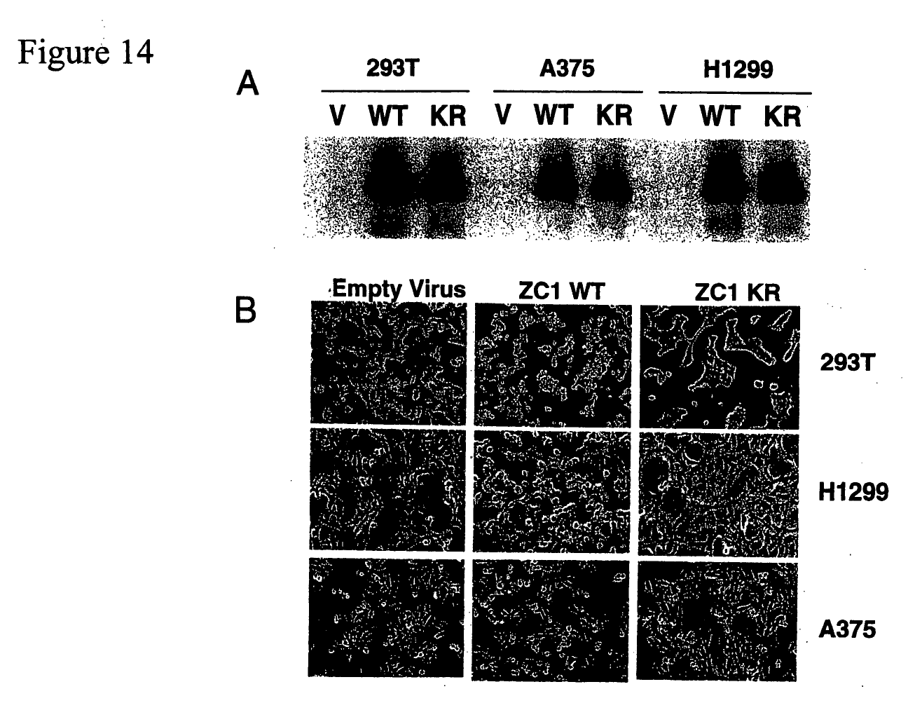

The morphology of 293T transformed embryonic kidney cells, H1299 lung carcinoma, and A375 melanoma cells were examined after infection with adenoviruses expressing wild type HGK, K54R HGK, or vector alone (no insert). In 293T, dramatic morphological changes are apparent (FIG. 14B, top panels). As compared with the vector control, the WT HGK infected cells are rounded up and more detached from each other as well as from the plate. In contrast, with infection with the K54R HGK virus, the cells become more adherent, and aggregated to form tightly associated colonies of cells. In H1299 (FIG. 14B, middle panels), similar trends were observed, with the WT HGK expressing cells showing a rounded morphology, and the KR HGK expressing cells appearing more flattened and spread out on the plate with prominent cell-cell junctions relative to the vector controls. In A375 cells (FIG. 14B, bottom panels), the trends are again similar, but less dramatic. This cell type also showed the lowest level of expression after infection (FIG. 14A).

The following reference documents also will help to provide a more complete understanding of the invention. Each of them is hereby incorporated by reference into the present disclosure.

Anastasiadis P. Z. et al. Inhibition of RhoA by p120 catenin. Nat. Cell Biol., 2:637-644 (2000).

Anastasiadis P. Z. & Reynolds A. B. Regulation of Rho GTPases by p120 catenin. Curr. Opin. Cell Biol. 13:604-610 (2001).

Bagrodia, S. & R. A. Cerione. Pak to the future. Trends Cell Biol. 9:350-355 (1999).

Bao, Q. & R. C. Hughes. Galectin-3 expression and effects on cyst enlargement and tubulogenesis in kidney epithelial MDCK cells cultured in three-dimensional matrices in vitro. J. Cell Sci. 108:2791-800 (1995).

Becker, E. et al. Nck-interacting Ste20 kinase couples Eph receptors to c-Jun N-terminal kinase and integrin activation. Mol. Cell Biol. 20:1537-45 (2000).

Berman, A. E. & N. I. Kozlova. Integrins: structure and functions. Membrane & Cell Biol. 13:207-44 (2000).

Biesova, Z. et al Isolation and characterization of e3B1, and eps8 binding protein that regulates cell growth. Oncogene 14(2): 233-241 (1997).

Bischoff, J. R. et al. A homologue of Drosophila aurora kinase is oncogenic and amplified in human colorectal cancers. EMBO J. 17:3052-65 (1998).

Boccaccio, C. et al. Induction of epithelial tubules by growth factor HGF depends on the STAT pathway. Nature 391:285-8 (1998).

Bowman, T. et al. STATs in oncogenesis. Oncogene 19:2474-88 (2000).

Bromberg, J. & J. E. Darnell Jr. The Role of STATs in transcriptional control and their impact on cellular function. Oncogene 19:2468-2473 (2000).

Caplan, S. et al. Human Vam6p promotes lysosome clustering and fusion in vivo. J. Cell Biol. 154:109-22 (2001).

Chadee, D. N. et al. Direct activation of mitogen-activated protein kinase kinase kinase MEKK1 by the Ste20p homologue GCK and the adapter protein TRAF2. Mol. Cell Biol. 22:737-49 (2002).

Comoglio, P. M. Pathway specificity for Met signaling. Nature Cell Biol. 3:E161-EI62 (2001).

Cox, A. D. & C. J. Der. Biological assays for cellular transformation. Methods Enzymol. 238:271-6 (1994).

Dai, Z. & A. M. Pendergast. Abi-2, a novel SH3-containing protein interacts with the c-Abl tyrosine kinase and modulates c-Abl transforming activity. Genes Dev. 9(21): 2569-2582 (1995).

Dai, Z. et al. Oncogenic Abl and Src tyrosine kinases elicit the ubiquitin-dependent degradation of target proteins through a Ras-independent pathway. Genes Dev. 12(10): 1415-1424 (1998).

Dan, I. et al. Molecular cloning of MINK, a novel member of mammalian GCK family kinases, which is up-regulated during postnatal mouse cerebral development. FEBS Lett. 469:19-23 (2000).

Dan, I. et al. The Ste20 group kinases as regulators of MAP kinase cascades. Trends Cell Biol. 11:220-30 (2001).

Davy, A. & S. M. Robbins. Ephrin-A5 modulates cell adhesion and morphology in an integrin-dependent manner. EMBO J. 19:5396-405 (2000).

Di Cunto, F. et al. Citron rho-interacting kinase, a novel tissue-specific ser/thr kinase encompassing the Rho-Rac-binding protein Citron. J. Biol. Chem. 273:29706-11 (1998).

Downing J. R. & Reynolds A. B. PDGF, CSF-1, and EGF induce tyrosine phosphorylation of p120, a pp60src transformation-associated substrate. Oncogene. 6(4):607-13 (1991).

Elion, E. A. Pheromone response, mating and cell biology. Current Opin. Microbiol. 3:573-81 (2000).

Florea, L. et al. A computer program for aligning a cDNA sequence with a genomic DNA sequence. Genome Res. 8:967-74 (1998).

Frisch, S. M. & H. Francis. Disruption of epithelial cell-matrix interactions induces apoptosis. J. Cell Biol. 124:619-26 (1994).

Frisch, S. M. & E. Ruoslahti. Integrins and anoikis. Current Opin. Cell Biol. 9:701-6 (1997).

Frisch, S. M. et al. Control of adhesion-dependent cell survival by focal adhesion kinase. J. Cell Biol. 134:793-9 (1996).

Fu, C. A. et al. TNIK, a novel member of the germinal center kinase family that activates the cJun N-terminal kinase pathway and regulates the cytoskeleton. J. Biol. Chem. 274:30729-37 (1999).

Giancotti, F. G. & E. Ruoslahti. Elevated levels of the alpha 5 beta 1 fibronectin receptor suppress the transformed phenotype of Chinese hamster ovary cells. Cell 60:849-859 (1990).

Giancotti, F. G. & E. Ruoslahti. Integrin Signaling. Science 285:1028-1032 (1999).

Grosheva I. et al. p120 catenin affects cell motility via moduation of activit of Rho-family GTPases: a link between cell-cell contact formation and regulation of cell locomotion. J. Cell Sci., 114:695-707 (2001).

Hungerford, J. E. et al. Inhibition of pp125FAK in cultured fibroblasts results in apoptosis. J. Cell Biol.135:1383-90 (1996).

Ishikawa, K. et al. Prediction of the coding sequence of unidentified human genes. X. The complete sequences of 100 new cDNA clones from brain which can code for large proteins in vitro. DNA Res. 5:169-176 (1998).

Juang, J. L. & F. M. Hoffmann. Drosophila abelson interacting protein (dAbi) is a positive regulator of abelson tyrosine kinase activity. Oncogene 18(37): 5138-5147 (1999).

Kanai-Azuma, M. et al. Nrk: a murine X-linked NIK (Nck-interacting kinase)-related kinase gene expressed in skeletal muscle. Mech. Dev. 89:155-9 (1999).

Kanner S. B. et al. Tyrosine phosphorylation of a 120-kilodalton pp60src substrate upon epidermal growth factor and platelet-derived growth factor receptor stimulation and in polyomavirus middle-T-antigen-transformed cells. Mol. Cell Biol. 11(2):713-20 (1991).

Kyriakis, J. M. & J. Avruch. Protein kinase cascades activated by stress and inflammatory cytokines. Bioessays 18:567-77 (1996).

Landau, N. R. & D. R. Littman. Packaging system for rapid production of murine leukemia virus vectors with variable tropism. J. Virol. 66:5110-5113 (1992).

Madaule, P. et al. Role of citron kinase as a target of the small GTPase Rho in cytokinesis. Nature 394:491-4 (1998).

Madaule, P. et al. A novel partner for the GTP-bound forms of rho and rae. FEBS Lett. 377:243-8 (1995).

Manser, E. et al. Expression of Constitutive Active alpha-PAK Reveals Effects of the Kinase on Actin and Focal Complexes. Mol. Cell. Biol. 17:1129-1143 (1997).

Miao, H. et al. Activation of EphA2 kinase suppresses integrin function and causes focal-adhesion-kinase dephosphorylation. Nature Cell Biol. 2:62-9 (2000).

Montesano, R. et al. Identification of a fibroblast-derived epithelial morphogen as hepatocyte growth factor. Cell 67:901-8 (1991).

Nakano, K. et al. NESK, a member of the germinal center kinase family that activates the c-Jun N-terminal kinase pathway and is expressed during the late stages of embryogenesis. J. Biol. Chem. 275:20533-9 (2000).

Nollet F. et al. The role of the E-cadherin/catenin adhesion complex in the development and progression of cancer. Mol. Cell Biol. Res. Commun. 2(2):77-85 (1999).

Noren N. K. et al. p120 catenin regulates the actin cytoskeleton via Rho family GTPases. J. Cell Biol., 150:567-580 (2000).

Paricio, N. et al. The Drosophila STE20-like kinase misshapen is required downstream of the Frizzled receptor in planar polarity signaling. EMBO J. 18:4669-78 (1999).

Poinat, P. et al. A Conserved Interaction between betal Integrin/PAT-3 and Nck-Interacting Kinase/MIG-15 that Mediates Commissural Axon Navigation in C. elegans. Curr. Biol. 12:622-31 (2002).

Pombo, C. M. et al. Activation of the SAPK pathway by the human STE20 homologue germinal centre kinase. Nature 377:750-4 (1995).

Qu, J., et al. Activated PAK4 Regulates Cell Adhesion and Anchorage-Independent Growth. Mol. Cell. Biol. 21:3523-3533 (2001).

Reynolds A. B. et al. Transformation specific tyrosine phosphorylation of a novel cellular protein in chicken cells expressing oncogenic variants of the avian cellular src gene. Mol. Cell. Biol., 9:629-638 (1989).

Reynolds A. B. et al. p120, a novel substrate of protein tyrosine kinase receptors and of p60 v-src, is related to cadherin-binding factors and b-catenin, plakoglobin and armadillo. Oncogene 7:2439-2445 (1992).

Ruan, W. et al. The SH2/SH3 adaptor protein dock interacts with the Ste20-like kinase misshapen in controlling growth cone motility. Neuron 24:595-605 (1999).

Scita, G. J. et al. EPS8 and E3B1 transduce signals from Ras to Rac. Nature 401 (6750):290-293 (1999).

Shi, Y. et al. Abl-interactor-1, a novel SH3 protein binding to the carboxy-terminal portion of the Abl protein, suppresses v-abl transforming activity. Genes Dev. 9(21):2583-2597 (1995).

Stella, M. C. & P. M. Comoglio. HGF: a multipfunctional growth factor controlling cell scattering. Int'l J. Biochem. & Cell Biol. 31:1357-1362 (1999).

Stradal, T. et al. The Abl interactor proteins localize to sites of actin polymerization at the tips of lamellipodia and filopodia. Curr. Biol. 11 (11):891-895 (2001).

Stuart, K. A. et al. Hepatocyte growth factor/scatter factor-induced intracellular signaling. Int. J. Exp. Path. 81:17-30 (2000).

Su, Y. C. et al. NIK is a new Ste20-related kinase that binds NCK and MEKK1 and activates the SAPK/JNK cascade via a conserved regulatory domain. EMBO J. 16:1279-90 (1997).

Su, Y. C. et al. The Ste20 kinase misshapen regulates both photoreceptor axon targeting and dorsal closure, acting downstream of distinct signals. Mol. Cell Biol. 20:4736-44 (2000).

Su, Y. C. et al. The Drosophila Ste20-related kinase misshapen is required for embryonic dorsal closure and acts through a JNK MAPK module on an evolutionarily conserved signaling pathway. Genes Dev. 12:2371-80 (1998).

Thoreson M. A. & Reynolds A. B. Altered expression of the catenin p120 in human cancer: implications for tumor progression. Differentiation 70:583-589 (2002).

Treisman, J. E. et al. misshapen encodes a protein kinase involved in cell shape control in Drosophila. Gene 186:119-25 (1997).

Trusolino, L. et al. A Signaling Adapter Function for a6b4 Integrin in the control of HGF-Dependent Invasive Growth. Cell 107:643-654 (2001).

Van Hengel J. et al. Nuclear localization of the p120(ctn) armadillo-like catenin is counteracted by a nuclear export signal and by E-cadherin expression. PNAS 96:7980-7985 (1999).

Wagner, S. et al. Association of the Ste20-like Kinase (SLK) with the Microtubule. J. Biol. Chem. 277:37685-37692 (2002).

Xue, H. et al. Attenuation of the expression of the focal adhesion kinase induces apoptosis in tumor cells. Blood 86:791-6 (1995).

Xue, Y. et al. Mesodermal patterning defect in mice lacking the Ste20NCK interacting kinase (NIK). Development 128:1559-72 (2001).

Yao, Z. et al. A novel human STE20-related protein kinase, HGK, that specifically activates the c-Jun N-terminal kinase signaling pathway. J. Biol. Chem. 274:2118-25 (1999).

Zhou. The Eph Family of Receptors and Ligands. Pharmacol. Ther. 77:151-181 (1998).

Zhu, X. & E. M. Hedgecock. Mig-15 encodes a novel ser/thr protein kinase of the Ste-20/p65PAK family. Worm Breeder's Gazette 14:76 (1996).

1. A method for determining whether a compound modulates ZC1 kinase activity in a mammal, comprising the steps of:
(a) measuring a base level of p120 catenin phosphorylation in a mammal,
(b) exposing said mammal to a compound, then
(c) measuring a post-exposure level of p120 catenin phosphorylation in said mammal, and
(d) comparing said base level with said post-exposure level,
wherein a difference in said base level and post-exposure level indicates that said compound modulates ZC1 kinase activity.

2. The method of claim 1, wherein said base and post-exposure levels of p120 catenin phosphorylation are measured in a tumor sample.

3. The method of claim 1, wherein a phospho-threonine specific antibody is used to measure p120 catenin phosphorylation.

4. The method of claim 3, wherein said phospho-threonine specific antibody specifically binds to p120 catenin.

5. A method for determining whether a compound modulates ZC1 kinase activity in a mammal, comprising:
(a) exposing a mammal to a compound, then
(b) measuring a post-exposure level of p120 catenin phosphorylation in said mammal, and
(c) comparing said post-exposure level of p120 catenin phosphorylation with a standard level of p120 catenin phosphorylation for said mammal,
wherein a difference in said post-exposure level and said standard level indicates that said compound modulates ZC1 kinase activity.

6. The method of claim 5, wherein a phospho-threonine specific antibody is used to measure p120 catenin phosphorylation.

7. The method of claim 6, wherein said phospho-threonine specific antibody specifically binds to p120 catenin.

8. A method of predicting whether a mammal will respond therapeutically to a method of treating cancer comprising administering at least one modulator of ZC1 kinase, wherein the method of predicting comprises the steps of:
(a) measuring the level of p120 catenin phosphorylation in a mammal with cancer, and
(b) comparing the level of p120 catenin phosphorylation in said mammal with cancer to a standard level of p120 catenin phosphorylation for said mammal,
wherein a difference in the level of p120 catenin phosphorylation in said mammal relative to a standard level of p120 catenin phosphorylation for said mammal indicates that said mammal will respond therapeutically to at least one modulator of ZC1 kinase.

9. The method of claim 8, wherein the level of p120 catenin phosphorylation in said mammal with cancer is measured in a tumor sample.

10. The method of claim 8, wherein a phospho-threonine specific antibody is used to measure p120 catenin phosphorylation.

11. The method of claim 10, wherein said phospho-threonine specific antibody specifically binds to p120 catenin.

12. A method for determining whether a compound modulates ZC3 kinase activity in a mammal, comprising the steps of:
(a) measuring a base level of Abi-1 phosphorylation in a mammal,
(b) exposing said mammal to a compound, then
(c) measuring a post-exposure level of Abi-1 phosphorylation in said mammal, and
(d) comparing said base level with said post-exposure level,
wherein a difference in said base level and post-exposure level indicates that said compound modulates ZC3 kinase activity.

13. The method of claim 12, wherein said base and post-exposure levels of Abi-1 phosphorylation are measured in a tumor sample.

14. The method of claim 12, wherein a phospho-threonine specific antibody is used to measure Abi-1 phosphorylation.

15. The method of claim 14, wherein said phospho-threonine specific antibody specifically binds to Abi-1

16. A method for determining whether a compound modulates ZC3 kinase activity in a mammal, comprising the steps of:
(a) exposing a mammal to a compound, then
(b) measuring a post-exposure level of Abi-1 phosphorylation in said mammal, and
(c) comparing said post-exposure level of Abi-1 phosphorylation with a standard level of Abi-1 phosphorylation for said mammal,
wherein a difference in said post-exposure level and said standard level indicates that said compound modulates ZC3 kinase activity.

17. The method of claim 16, wherein a phospho-threonine specific antibody is used to measure Abi-1 phosphorylation.

18. The method of claim 17, wherein said phospho-threonine specific antibody specifically binds to Abi-1.

19. A method of predicting whether a mammal will respond therapeutically to a method of treating cancer comprising administering at least one modulator of ZC3 kinase, wherein the method of predicting comprises the steps of:
(a) measuring the level of Abi-1 phosphorylation in a mammal with cancer, and
(b) comparing the level of Abi-1 phosphorylation in said mammal with cancer to a standard level of Abi-1 phosphorylation for said mammal,
wherein a difference in the level of Abi-1 phosphorylation in said mammal relative to a standard level of Abi-1 phosphorylation for said mammal indicates that said mammal will respond therapeutically to at least one modulator of ZC3 kinase.

20. The method of claim 19, wherein the level of Abi-1 phosphorylation in said mammal with cancer is measured in a tumor sample.

21. The method of claim 19, wherein a phospho-threonine specific antibody is used to measure Abi-1 phosphorylation.

22. The method of claim 21, wherein said phospho-threonine specific antibody specifically binds to Abi-1.
